(19) United States
(12) Patent Application Publication
(10) Pub. No.: US 2018/0330808 A1
(43) Pub. Date: Nov. 15, 2018

(54) Machine learning system for disease, patient, and drug co-embedding, and multi-drug recommendation

(71) Applicant: Petuum Inc., Pittsburgh, PA (US)

(72) Inventors: Pengtao Xie, Pittsburgh, PA (US); Eric Xing, Pittsburgh, PA (US)

(21) Appl. No.: 15/946,482

(22) Filed: Apr. 5, 2018

Related U.S. Application Data
(60) Provisional application No. 62/504,474, filed on May 10, 2017.

(51) Int. Cl. G16H 20/10; G16H 10/60; G06N 7/00

(52) U.S. Cl. CPC G16H 20/10; G06N 7/005; G16H 10/60

(57) ABSTRACT

A medication-recommending system is disclosed. The medication-recommendation system includes: a medication-medication correlation (MMC) sub-module configure to generate a correlation score of a first candidate medication and a second candidate medication; a medication-EHR dependency (MED) sub-modules configure to generate a dependency score between each of the first and second medications and an electronic health record (EHR); a relation-constraint (RC) sub-module configured to generate a relationship constraint indicating the interaction relation between the first and second medications; and a medication selection (MS) sub-module configure to select one or more recommended medications from at least the first and second medications based on the correlation score, dependency scores, and relational constraint.

### FIELD OF THE INVENTION

The present invention generally relates to Machine Learning (ML) for healthcare, and more particularly, is directed to a method and system of performing medication recommendation via a relation-constrained subset selection model.

### BACKGROUND

Prescribing medications is a complicated process, where several aspects need to be taken into consideration. First and foremost, what medications can be used for treatment of one or more diagnosed diseases? For a single disease, there can be hundreds of medications for treatment. Typically, a patient has multiple conditions/diseases simultaneously, which further increases the number of candidate medications that can be prescribed to the patient. Second, some medications have adverse interactions and are discouraged to be used together. Physicians need to keep these antagonistic medications in mind and avoid prescribing them simultaneously. The number of medication pairs that have antagonistic interactions is very large, which makes it highly challenging to remember all of them precisely. Third, in clinical practice, rich knowledge has been accumulated so as to identify that, when used together, some medications can generate a synergy benefit and treat a disease more effectively. Such knowledge should be leveraged to improve the treatment recommendation. The number of synergy relations is large as well, making it difficult to remember and use. It is highly challenging for physicians to clearly remember the vast amount of knowledge mentioned-above (e.g., what drugs can be utilized to treat a certain disease; which drugs have adverse interactions or synergy relations). Because a patient can be diagnosed with several diseases, how to select from a large number of drugs that can be potentially applied to treat these diseases a small subset that possess the best treatment effect while avoiding adverse interaction and promoting synergy benefit becomes even more difficult.

Artificial Intelligence (AI) systems have become increasingly popular in clouds and data centers, especially in an enterprise environment. These systems are designed to resolve complicated issues involving large amount of data through, for example, self-learning. A need for an Operating System (OS) software in the enterprise AI data centers that can manage all the assets mentioned above with regard to providing medication for a particular medical condition is desired.

### SUMMARY OF THE INVENTION

The presently disclosed embodiments are directed to solving issues relating to one or more of the problems presented in the prior art, as well as providing additional features that will become readily apparent by reference to the following detailed description when taken in conjunction with the accompanying drawings.

One embodiment is directed to a medication-recommending system. The medication-recommending system includes: a medication-medication correlation (MMC) sub-module configure to generate a correlation score of a first candidate medication and a second candidate medication; a medication-EHR dependency (MED) sub-modules configure to generate a dependency score between each of the first and second medications and an electronic health record (EHR); a relation-constraint (RC) sub-module configured to generate a relationship constraint indicating the interaction relation between the first and second medications; and a medication selection (MS) sub-module configure to select one or more recommended medications from at least the first and second medications based on the correlation score, dependency scores, and relational constraint.

Another embodiment is directed to a method of recommending medications. The method includes: receiving an electronic health record (EHR) including a plurality of modalities; encoding each of the modalities into a vector representation; combining the vector representations into a single vector; receiving profile articles of a plurality of candidate medications; encoding the profile articles into article vectors; computing a dependency score between the EHR and each candidate medication based on the single vector and the article vectors; computing a correlation score between a pair of medications of the plurality of candidate medications based on the article vectors; combining the dependency score and the correlation score into a kernel matrix generating at least one binary constraint based on medication interactions among the plurality of candidate medications; and selecting a subset of the plurality of the candidate medications based on the kernel matrix and the at least one binary constraint.

Further features and advantages of the present disclosure, as well as the structure and operation of various embodiments of the present disclosure, are described in detail below with reference to the accompanying drawings.

### BRIEF DESCRIPTION OF THE DRAWINGS

The present disclosure, in accordance with one or more various embodiments, is described in detail with reference to the following figures. The drawings are provided for purposes of illustration only and merely depict exemplary embodiments of the disclosure. These drawings are provided to facilitate the reader's understanding of the disclosure and should not be considered limiting of the breadth, scope, or applicability of the disclosure. It should be noted that for clarity and ease of illustration these drawings are not necessarily made to scale.

FIG. 1 is a block diagram illustrating the exemplary modules of a Medication Recommendation (MR) system, according to embodiments of the invention;

FIG. 2 is a block diagram illustrating the exemplary modules of the Electronic Health Record (EHR) encoding sub-module of the MR system of FIG. 1, according to embodiments of the invention;

FIG. 3 is a flowchart diagram illustrating the exemplary steps in a process that can be carried out by the MR system of FIG. 1, according to embodiments of the invention; and

FIG. 4 is a block diagram illustrating the exemplary modules of a computer system running the MR system of FIG. 1.

### DETAILED DESCRIPTION OF EXEMPLARY EMBODIMENTS

The following description is presented to enable a person of ordinary skill in the art to make and use the invention. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the invention. Thus, embodiments of the present invention are not intended to be limited to the examples described herein and shown, but is to be accorded the scope consistent with the claims.

The word “exemplary” is used herein to mean “serving as an example or illustration.” Any aspect or design described herein as “exemplary” is not necessarily to be construed as preferred or advantageous over other aspects or designs.

Reference will now be made in detail to aspects of the subject technology, examples of which are illustrated in the accompanying drawings, wherein like reference numerals refer to like elements throughout.

It should be understood that the specific order or hierarchy of steps in the processes disclosed herein is an example of exemplary approaches. Based upon design preferences, it is understood that the specific order or hierarchy of steps in the processes may be rearranged while remaining within the scope of the present disclosure. The accompanying method claims present elements of the various steps in a sample order, and are not meant to be limited to the specific order or hierarchy presented.

Embodiments disclosed herein are directed to a Medication Recommendation (MR) system designed for recommending medication based on one or more sets of data including, but not limited to the patient's health records, profiles of medications, correlations between medications and patients' symptoms and/or diagnosis, and known interactions between the different medications.

In one embodiment, such an MR system is configured to perform medication recommendation tasks by receiving inputs of an electronic health records (EHRs) (e.g., the clinical notes, lab test values, physical exams, medical images, etc.), profile articles of medications (e.g., in Drugs.com which is an encyclopedia of medications, each medication has an article describing what this medication is, what diseases/conditions it can treat, how it should be taken, its side effect, etc.) and interaction relations (e.g., diflucan and dolasetron have an antagonistic interaction; but aspirin and clopidogre have a synergistic interaction) between medications and generating an output of a subset of medications that can best treat this patient. The MR system utilizes deep neural networks to learn representations for patients' EHRs and medications, and compute the correlation among medications and the dependency between EHRs and medications. It uses a structural probabilistic model to perform medication-subset selection and is able to capture medication-correlation of any order. The MR system can flexibly incorporate the interaction relations among medications for better recommendation.

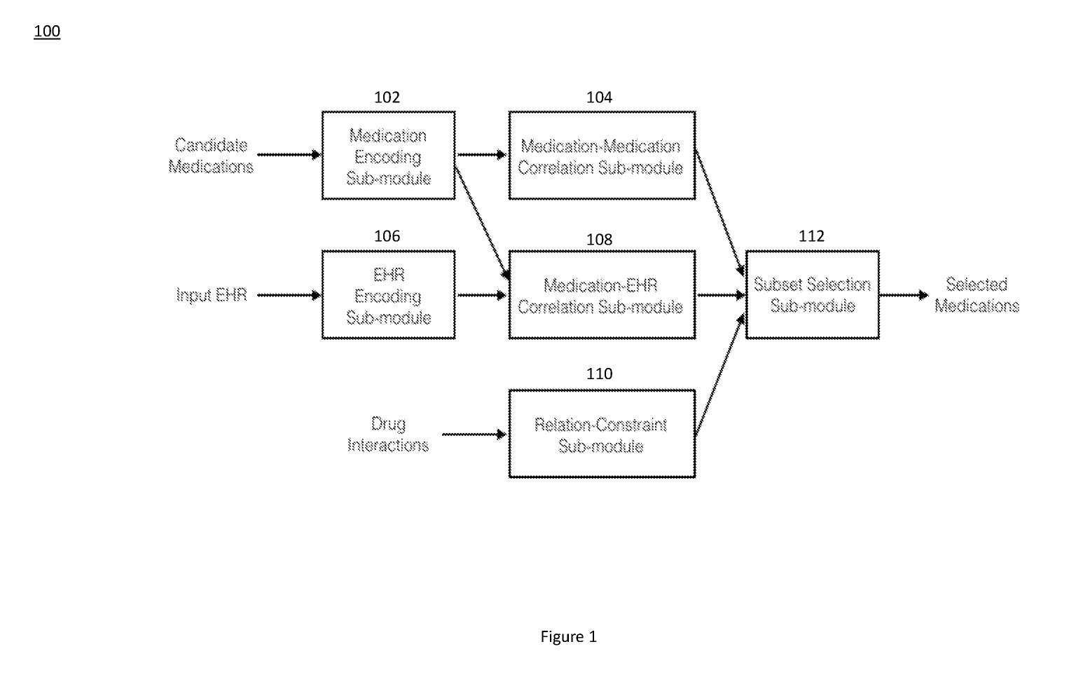

FIG. 1 illustrates an exemplary MR system 100, according to an embodiment of the invention. As shown in FIG. 1, the MR system 100 can include a Medication Encoding (ME) Sub-module 102, a Medication-Medication Correlation (MMC) Sub-module 104, an Electronic Health Records (EHR) Encoding Sub-module 106, a Medication-EHR Correlation (MEC) Sub-module 108, a Relation-Constraint (RC) Sub-module 110, and a Subset Selection (SS) Sub-Module 112. The ME Sub-module 104 can receive profiles of various medications. The profiles can be articles provided by an external resource such as Drugs.com, which is an encyclopedia of medications. Each article can describe what the medication is, the diseases/conditions it can treat, direction of use, dosage, side effects, etc.

The ME Sub-module 102 can take the information on the medications as input and produce a vector representation of this medication. In one example, specifically, the ME sub-module 104 can be a convolutional neural network which takes the word sequence of a medication's profile article (e.g., a Drugs.com article) as input, performs convolution, pooling, and generates a vector representing this article. The vector can be the bag-of-words feature vector of the medications' profile articles.

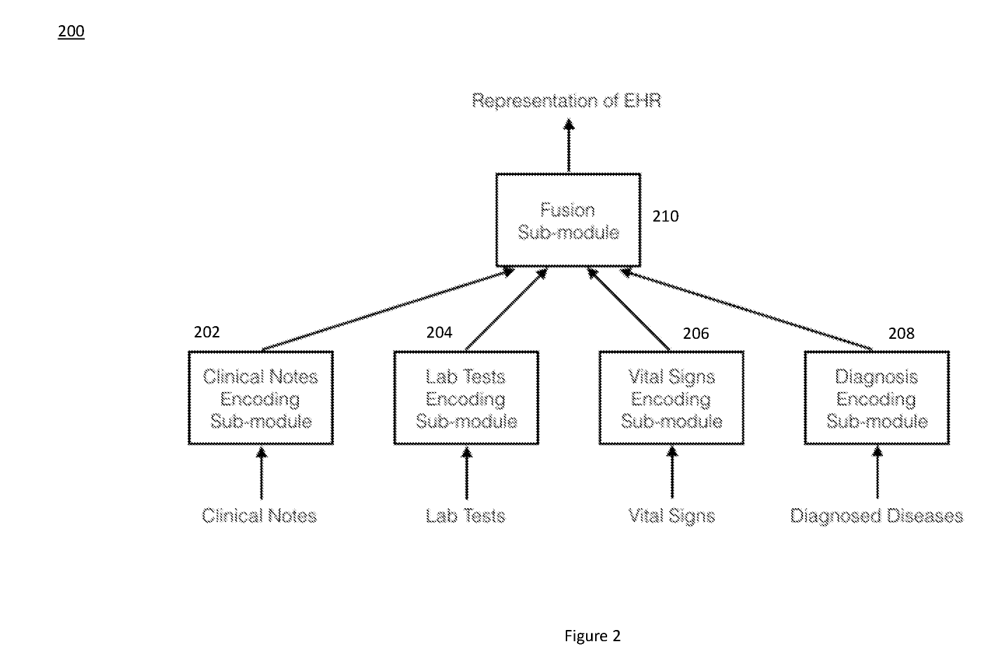

In this embodiment, the EHR encoding (EE) sub-module 106 of the MR system 100 is configured to receive and learn feature representations of electronic health records (EHR), which can include multiple modalities of clinical information, including clinical notes, lab tests, vital signs, demographics, etc. The EE Sub-module 106 is discussed in detail below with reference to FIG. 2.

As illustrated in FIG. 2, in one embodiment, the EE Sub-module 106 can include four encoding sub-modules that encode four modalities of data. These four encoding sub-modules include a Clinical Notes Encoding Sub-module 202 for encoding clinic notes, a Lab Tests Encoding Sub-module 204 for encoding lab test information, a Vital Signs Encoding Sub-module 206 for encoding vital sign information, and a Diagnosis Encoding Sub-module 208 for encoding diagnosis information. It should be understood that additional encoding modules can be included for encode other types of EHR information. Each of the Clinical Notes Encoding Sub-module 202, Lab Tests Encoding Sub-modules 204, Vital Signs Encoding Sub-module 206, and Diagnosis Encoding Sub-module can be connected to a Fusion sub-module 210 that can combine the representations of individual modalities (e.g., clinical notes, lab tests, vital signs, and diagnosis) into a holistic one. In one example, this fusion can be undertaken by a feedforward neural network that takes the representation vectors of the four data modalities as inputs and outputs a vector as the holistic representation of the entire EHR. The representation vector can be the bag-of-words feature vector of clinical notes. The clinical notes encoding sub-module can be a convolutional neural network that is able to capture the local correlations among adjacent words and long-range semantics. The lab tests and vital signs encoding sub-module can be long short-term memory networks that are able to capture the temporal structure among lab tests and vital signs. The diagnosis-encoding sub-module can be a feedforward network that captures non-linear relations among diseases.

Referring back to FIG. 1, the MMC sub-module 104 can measure the correlation of two medications. The MMC sub-module 104 can take the vector representations that are generated by the ME sub-module 102 of the two medications as inputs and produce a score (e.g., Pearson correlation score) indicating the strength of correlation between the two medications. In one embodiment, the MMC sub-module can be a feedforward neural network. The two medications' vectors can be concatenated and fed into this network. The network can perform a few successive nonlinear transformations of the concatenated vector and output a scalar that measures medication-correlation.

The MEC sub-module 108 can measure the dependency between a medication and an EHR. As illustrated in FIG. 1, the MEC sub-module 108 can take the vector representation (produced by the ME sub-module 102) of the medication and the representation (produced by the EE sub-module 106) of the EHR as inputs and produce a score (e.g., the cosine similarity) indicating the strength of dependency between the medication and the EHR. The MEC sub-module can be parameterized by a feedforward deep neural network. The representation vector of the medication and the vector of the EHR can be concatenated and inputted into the network. In turn, the network can perform a few successive nonlinear transformations of the concatenated vector and produce a scalar score that measure medication-EHR dependency. The terms MEC Sub-module and the term Medication EHR Dependency (MED) Sub-module are used interchangeably in this application.

The RC sub-module can use the interaction relations between medications to control the selection of medications. The relations can have two types. If the interaction is antagonistic, the two medications are prohibited to be co-selected to treat a disease. If the interaction is synergic, the two medications are encouraged to be co-selected. These antagonism and synergy relations can be obtained from one or more existing external medical knowledge bases. In one embodiment, the interaction can be represented as a binary constraint. The term RC Sub-module and the term Medication Interaction Procession (MIP) Sub-module are used interchangeably in this application.

The SS sub-module 110 (or Medication Selection (MS) sub-module) can select a subset of medications from the candidate medications, as the prescription to patients. In one example, the SS sub-module 110 can take the following information as inputs: (1) correlation scores between the medications that are produced by the MMC sub-module 104; (2) dependency scores between the medications and the input EHR that are produced by the MEC sub-module 108; (3) relational constraints regarding medication co-selection that are produced by the RC sub-module 110. Also, the SS sub0module 110 can produce a subset of medications that maximize the correlations scores and dependency scores but do not violate the constraints. At the core of this sub-module 110 is a probabilistic model. In one embodiment, the probabilistic model can be referred to as Determinantal Point Process (DPP) that is able to capture the medication-medication correlation of any order. In one embodiment, DPP can be a stochastic process defined on subsets. Given a set of medications {aᵢ}ᵢ₌₁^{K}, each represented by a vector aᵢ, DPP computes a K-by-K kernel matrix L, where Lᵢⱼ=k(aᵢ, aⱼ) and k(.,.) is a kernel function. Then the probability over a subset of medications S⊆{1, . . . , K} can be defined as:

$$p ( ) = \frac{det ( {L}_{} )}{det ( L + I )}$$

where L_{S} is the submatrix of L indexed by element in S, I is an identity matrix and det(⋅) denotes the determinant of a matrix.

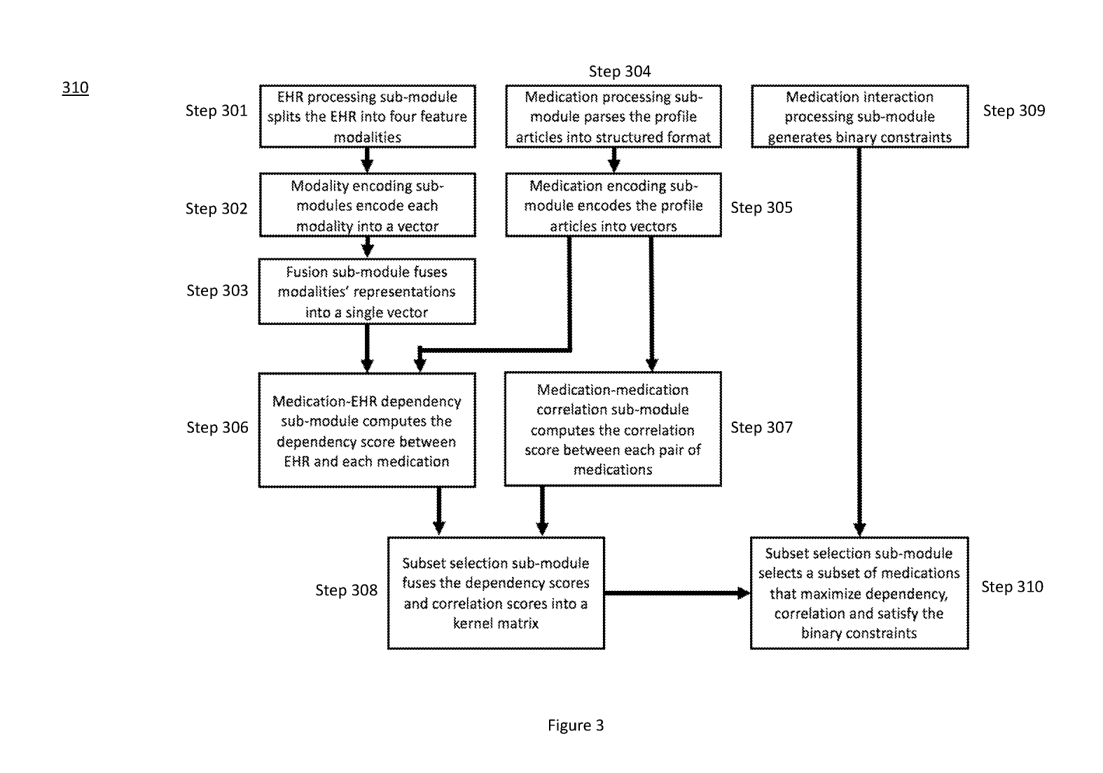

FIG. 3 illustrates the exemplary steps performed by the MR system 100 of FIG. 1 when selecting medications to treat a particular disease. First, the EHR processing sub-module of the MR system splits the EHR into four feature modalities (step 301). Each of the modalities can then be encoded into a vector by a corresponding Modality Encoding Sub-module (e.g., one of the Clinical Notes Encoding sub-module 202, Lab Tests Encoding sub-module 204, Vital Signs Encoding sub-Module 206, and Diagnosis Encoding sub-Module 208 of FIG. 2) of the EHR Encoding sub-module (step 302). Thereafter, the Fusion sub-module of the EHR Encoding sub-module can fuse the vector representations of the four modalities into a single vector (step 303). It should be understood that in various embodiments, the number of modalities can be different than four. There may be additional modalities not explicitly discussed herein. A Medication Processing sub-module can parse profile articles of candidate medications into a structured format (step 304). The ME Sub-module can then encode the profile articles into vectors (step 305). The MED Sub-module can compute a dependency score between the EHR and each medication using the output from steps 303 and 305 (step 306). The MMC Sub-module can compute the correlation score between a pair of medications using the output from step 305 (step 307). The SS Sub-module can then fuse the dependency scores from step 306 and the correlation scores from step 307 into a kernel matrix (step 308). The MIP Sub-module can generate binary constraints based medication interactions (step 309). The output from steps 308 and 309 can be used by the SS Sub-module to select a subset of the candidate medications than can maximize dependency, correlation and satisfy the binary constraints.

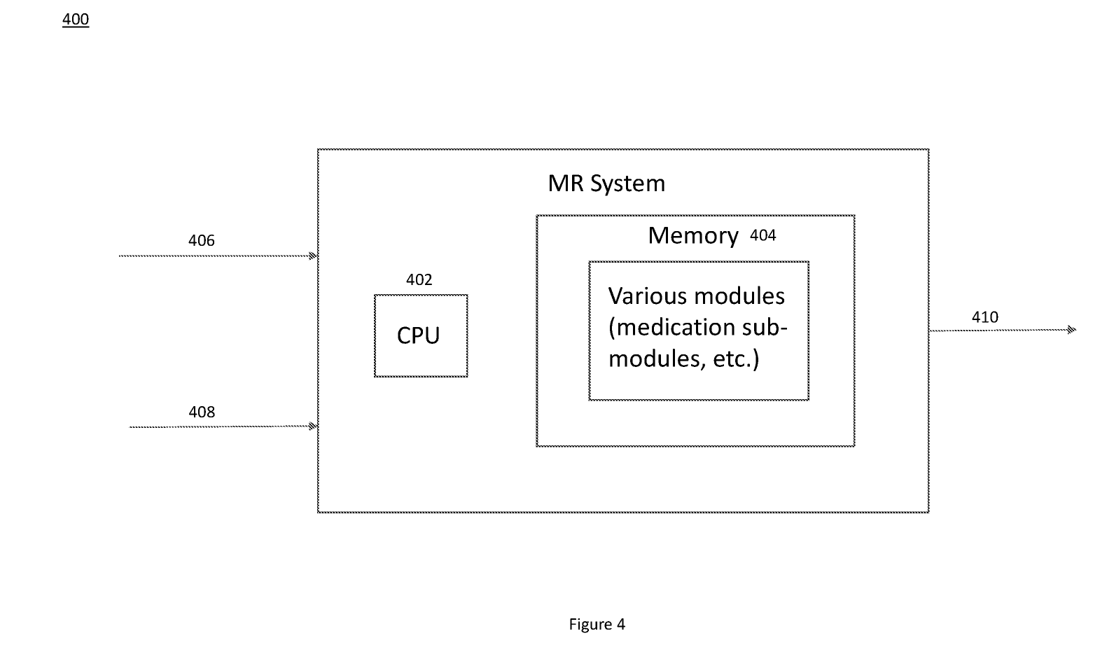

The MR System 100 of FIG. 1 can be implemented on a computer system such as the one shown in FIG. 4. The computer system 400 can include, for example, a central processing unit (CPU) 402 and a computer-readable medium such as a memory 404. The memory 404 can store the various modules and sub-modules such as those shown in FIGS. 1 and 2. When executed by the CPU 402, the various modules can perform the steps to recommend medications as described above with reference to FIG. 3. The system 400 can receive external data such as candidate medication, input EHR, and/or drug interactions, through one or more input ports 406, 408. The system 400 can also include at least one output 410 for outputting medication recommendations.

While various embodiments of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various exemplary embodiments and implementations, it should be understood that the various features and functionality described in one or more of the individual embodiments are not limited in their applicability to the particular embodiment with which they are described. They instead can be applied alone or in some combination, to one or more of the other embodiments of the disclosure, whether or not such embodiments are described, and whether or not such features are presented as being a part of a described embodiment. Thus the breadth and scope of the present disclosure should not be limited by any of the above-described exemplary embodiments.

In this document, the term “module” as used herein, refers to software, firmware, hardware, and any combination of these elements for performing the associated functions described herein. Additionally, for purpose of discussion, the various modules are described as discrete modules; however, as would be apparent to one of ordinary skill in the art, two or more modules may be combined to form a single module that performs the associated functions according embodiments of the invention.

In this document, the terms “computer program product”, “computer-readable medium”, and the like, may be used generally to refer to media such as, memory storage devices, or storage unit. These, and other forms of computer-readable media, may be involved in storing one or more instructions for use by processor to cause the processor to perform specified operations. Such instructions, generally referred to as “computer program code” (which may be grouped in the form of computer programs or other groupings), when executed, enable the computing system.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; and adjectives such as “conventional,” “traditional,” “normal,” “standard,” “known”, and terms of similar meaning, should not be construed as limiting the item described to a given time period, or to an item available as of a given time. But instead these terms should be read to encompass conventional, traditional, normal, or standard technologies that may be available, known now, or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to”, or other like phrases in some instances shall not be read to mean that the narrower case is intended or required in instances where such broadening phrases may be absent.

Additionally, memory or other storage, as well as communication components, may be employed in embodiments of the invention. It will be appreciated that, for clarity purposes, the above description has described embodiments of the invention with reference to different functional units and processors. However, it will be apparent that any suitable distribution of functionality between different functional units, processing logic elements or domains may be used without detracting from the invention. For example, functionality illustrated to be performed by separate processing logic elements or controllers may be performed by the same processing logic element or controller. Hence, references to specific functional units are only to be seen as references to suitable means for providing the described functionality, rather than indicative of a strict logical or physical structure or organization.

Furthermore, although individually listed, a plurality of means, elements or method steps may be implemented by, for example, a single unit or processing logic element. Additionally, although individual features may be included in different claims, these may possibly be advantageously combined. The inclusion in different claims does not imply that a combination of features is not feasible and/or advantageous. Also, the inclusion of a feature in one category of claims does not imply a limitation to this category, but rather the feature may be equally applicable to other claim categories, as appropriate.

What is claimed is:

1. A medication-recommending system comprising:
a medication-medication correlation (MMC) sub-module configured to generate a correlation score of a first candidate medication and a second candidate medication;
a medication-EHR dependency (MED) sub-module configured to generate a dependency score between each of the first and second medications and an electronic health record (EHR);
a relation-constraint (RC) sub-module configured to generate a relationship constraint indicating the interaction relation between the first and second medications; and
a medication selection (MS) sub-module configure to select one or more recommended medications from at least the first and second medications based on the correlation score, dependency scores, and relational constraint.

2. The system of claim 1, further comprising an electronic health record (EHR) encoding (EE) sub-module configured to generate a representation of an EHR.

3. The system of claim 2, wherein the representation of an EHR comprises a vector representation.

4. The system of claim 2, wherein the EE sub-module further comprises at least one of:
a clinical notes encoding sub-module configured to encode a clinical note;
a lab testing encoding sub-module configured to encode a lab test;
a vital signs encoding sub-module configured to encode a vital sign; and
a diagnosis encoding sub-module configured to encode a diagnosis.

5. The system of claim 4, further comprising a fusion sub-module configured to combine at least two of the encoded clinical note, encoded lab test, encoded vital sign, and encoded diagnosis.

6. The system of claim 5, wherein an output of the fusion sub-module comprises a representation of the EHR.

7. The system of claim 2, wherein an EHR comprises at least one of a clinical note, a lab test value, a physical exam, and a medical image.

8. The system of claim 1, further comprising a medication encoding (ME) sub-module configured to generate a representation for each of the first and second medications.

9. The system of claim 8, wherein the representation of each of the first and second medications comprises a vector representation.

10. The system of claim 8, wherein each of the first and second medications comprises a profile article of the medication.

11. The system of claim 1, wherein the relationship constraint indicating the interaction relation between the first and second medications can be a binary constraint indicating whether the interaction relation is either antagonistic or synergic.

12. The system of claim 8, wherein the ME sub-module comprises a convolutional neural network configured to take a word sequence of a medication's profile article as input, perform convolution, pooling and generate a vector representing the profile article.

13. The system of claim 1, wherein the MMC sub-module comprises a feedforward neural network configured to receive two medications' concatenated vectors, perform at least one nonlinear transformation of the concatenated vectors, and output a scalar that measures medication-correlation.

14. The system of claim 13, wherein the scalar comprises a Pearson correlation score.

15. The system of claim 1, wherein the MED sub-module is parameterized by a feedforward deep neural network that receives concatenated representation vectors of a medication and an EHR, and performs at least one nonlinear transformation of the concatenated representation vectors.

16. The system of claim 1, wherein the MS sub-module is configured to use a probabilistic model.

17. The system of claim 16, wherein the probabilistic model comprises a Determinantal Point Process (DPP).

18. The system of claim 1, wherein the dependency score comprises a cosine similarity.

19. A computer-readable medium storing instructions, when executed by a processor, performs a method of recommending medications, comprising:
receiving an electronic health record (EHR) including a plurality of modalities;
encoding each of the modalities into a vector representation;
combining the vector representations into a single vector;
receiving profile articles of a plurality of candidate medications;
encoding the profile articles into article vectors;
computing a dependency score between the EHR and each candidate medication based on the single vector and the article vectors;
computing a correlation score between a pair of medications of the plurality of candidate medications based on the article vectors;
combining the dependency score and the correlation score into a kernel matrix
generating at least one binary constraint based on medication interactions among the plurality of candidate medications; and
selecting a subset of the plurality of the candidate medications based on the kernel matrix and the at least one binary constraint.
